(19) United States
(12) Patent Application Publication
(10) Pub. No.: US 2011/0003862 A1
(43) Pub. Date: Jan. 6, 2011

(54) 3-Animopyraolines for Treatment of Neurodegenerative and Psychiatric Diseases

(75) Inventors: Philip E. Brandish, North Wales, PA (US); Nicholas Brandon, Princeton, NJ (US); Alister Campbell, London (GB); Timothy Sparey, London (GB)

Correspondence Address: MERCK, P O BOX 2000, RAHWAY, NJ, 07065-0907, US

(73) Assignees: MERCK SHARP & DOHME LIMITED, Hertfordshire (GB); MERCK & CO., INC., Rahway, NJ (US)

(21) Appl. No.: 12/224,077

(22) PCT Filed: Feb. 15, 2007
(86) PCT No.: PCT/GB2007/050065

§ 371 Date: Sep. 17, 2010

(30) Foreign Application Priority Data
Feb. 17, 2006 (GB) 0603181.9

(51) Int. Cl. A61K 31/4439; C07D 231/06; A61K 31/415; C07D 401/02; A61P 25/28

(52) U.S. Cl. 514/341; 548/371.4; 514/407; 546/275.4

(57) ABSTRACT

Compounds of the formula (I), inhibit D-amino acid oxidase and hence find use in treating degenerative and psychiatric diseases.

The present invention relates to the use of substituted pyrazolines for the treatment of neurodegenerative and psychiatric diseases and disorders, either as a monotherapy or in combination with a further agent useful for the treatment of such diseases and disorders.

N-methyl-D-aspartate (NMDA)-glutamate receptors are expressed at excitatory synapses throughout the central nervous system (CNS). These receptors mediate a wide range of brain processes, including synaptic plasticity associated with certain forms of memory formation and learning. NMDA-glutamate receptors require binding of two agonists to effect neurotransmission. One of these agents is the excitatory amino acid L-glutamate, while the second agonist is thought to be D-serine. In animals D-serine is synthesized from L-serine by serine racemase and degraded to its corresponding keto acid by D-amino acid oxidase. Together, serine racemase and D-amino acid oxidase are thought to play a crucial role in modulating NMDA receptor mediated neurotransmission by regulating CNS concentrations of D-serine. D-amino acid oxidase inhibitors may also modulate other D-amino acid oxidase substrates providing therapeutic activity independent of NMDA receptor activation.

Several studies have linked D-amino acid oxidase (DAO) activity with psychiatric disorders such as schizophrenia and bipolar disorder, for example Chumakov et al, PNAS, 2002, 99, 13675-80; Schumacher et al, Mol. Psychiatry, 2004, 9, 203-7; Liu et al, Neurosci. Lett., 2004, 369, 228-33; and Kapoor et al, Brain Res., 2006, 1106, 205-10. Similarly, several patent publications disclose classes of compounds which are said to be useful for treating psychiatric and/or neurodegenerative diseases via inhibition of DAO, for example WO 03/039540; US 2005/0143443; and US 2005/0143434, but there is no disclosure of 3-aminopyrazolines for this purpose.

EP 0 070 376 and EP 127 371 disclose 3-aminopyrazoline derivatives having anti-inflammatory properties, but make no mention of DAO inhibition or utility in treating psychiatric and/or neurodegenerative disorders.

It has now been discovered that a group of substituted pyrazolines have activity as D-amino acid oxidase inhibitors and are useful in the treatment of neurodegenerative and psychiatric disorders and diseases.

Accordingly, in a first aspect, the present invention is directed to the use of a compound of the formula (I):

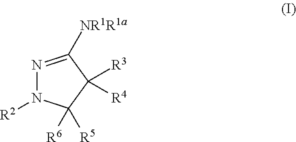

or a pharmaceutically acceptable salt thereof, wherein

R¹ and R^{1a} are independently selected from hydrogen, C₁₋₆alkyl, haloC₁₋₆alkyl, hydroxyC₁₋₆alkyl, C₁₋₆alkylcarbonyl, and a group SO₂R⁷ wherein R⁷ is amino or C₁₋₆alkyl optionally substituted by phenyl;

R² is selected from phenyl, 5- or 6-membered heteroaryl which is optionally benzofused, 5- or 6-membered carbocyclic, and 5- or 6-membered heterocyclic containing at least one hetero atom selected from oxygen, nitrogen and sulphur, each of which may be substituted by C₁₋₆alkyl, C₁₋₆alkoxy, CF₃ or OCF₃;

or R² is C₁₋₆alkyl optionally substituted by hydroxyl, halo or amino optionally substituted by one or two C₁₋₆alkyl groups; or a group (CH₂)ₘphenyl, wherein m is 1 or 2, which may be substituted by halo, hydroxyl or amino optionally substituted by one or two C₁₋₆allyl groups;

and R³, R⁴, R⁵ and R⁶ are independently selected from hydrogen, CF₃, C₁₋₆allyl and phenyl each optionally substituted by halo or hydroxyl;

for the manufacture of a medicament for treatment of a neurodegenerative or psychiatric condition associated with D-amino acid oxidase.

In a further aspect the present invention provides a compound of the formula (I) as defined above for use in treatment of a neurodegenerative or psychiatric disease associated with D-amino acid oxidase.

In a further aspect, the present invention provides a method of treatment of neurodegenerative and psychiatric diseases associated with D-amino acid oxidase by the administration of an effective amount of a compound of the formula (I), as herein defined.

In one embodiment, at least one of R′ and lea is hydrogen. In a second embodiment both R′ and lea are hydrogen.

In a further embodiment, when R² is aromatic, this is selected from a phenyl, pyridyl and pyrazolyl ring, in particular phenyl. When R² is a saturated ring, this is preferably cyclohexyl. Suitable substituents for the aromatic rings include halo, particularly fluoro and chloro, methoxy, amino or trifluoromethyl. Suitably, R² is phenyl optionally substituted by fluoro, chloro, methoxy, or trifluoromethyl; unsubstituted pyridyl; or pyrazolyl substituted by amino.

In a further embodiment, R³, R⁴, R⁵ and R⁶ are independently selected from hydrogen, CF₃, C₁₋₆alkyl and phenyl, each optionally substituted by halo. Preferably at most two of R³, R⁴, R⁵ and R⁶ are other than hydrogen. Preferably at least one of R³ and R⁴ is hydrogen. Preferred values for R³, R⁴, R⁵ and R⁶ include hydrogen, methyl, ethyl, trifluoromethyl and phenyl optionally substituted by a fluoro or chloro atom which is suitably at the para position. When one of R³, R⁴, R⁵ and R⁶ is optionally substituted phenyl, the others are preferably hydrogen.

Preferred compounds of the formula (I) include:
5-(4-chlorophenyl)-1-(2-fluorophenyl)-4,5-dihydro-1H-pyrazol-3-amine,
5-(4-chlorophenyl)-1-(2-fluorophenyl)-4,5-dihydro-1H-pyrazol-3-amine,
1-(3-fluorophenyl)-5-methyl-4,5-dihydro-1H-pyrazol-3-amine,
1-(2-fluorophenyl)-5-phenyl-4,5-dihydro-1H-pyrazol-3-amine,
1-(3-chlorophenyl)-5-methyl-4,5-dihydro-1H-pyrazol-3-amine,
1-(4-chlorophenyl)-5-methyl-4,5-dihydro-1H-pyrazol-3-amine,
5-ethyl-1-(2-fluorophenyl)-4,5-dihydro-1H-pyrazol-3-amine,
1-(3-chlorophenyl)-4-methyl-4,5-dihydro-1H-pyrazol-3-amine,
1-(3-fluorophenyl)-4,5-dihydro-1H-pyrazol-3-amine,
5-(4-fluorophenyl)-1-pyridin-3-yl-4,5-dihydro-1H-pyrazol-3-amine,
1-(4-chlorophenyl)-5-phenyl-4,5-dihydro-1H-pyrazol-3-amine,
1-(4-chlorophenyl)-5-phenyl-4,5-dihydro-1H-pyrazol-3-amine,
1-(3-chlorophenyl)-5-phenyl-4,5-dihydro-1H-pyrazol-3-amine,
1-(2-fluorophenyl)-4-methyl-4,5-dihydro-1H-pyrazol-3-amine,
1-(3-chlorophenyl)-4,5-dihydro-1H-pyrazol-3-amine,
4-methyl-1-pyridin-2-yl-4,5-dihydro-1H-pyrazol-3-amine,
5-ethyl-1-pyridin-2-yl-4,5-dihydro-1H-pyrazol-3-amine,
1-(3-fluorophenyl)-5-(trifluoromethyl)-4,5-dihydro-1H-pyrazol-3-amine,
1-(2-fluorophenyl)-4,5-dihydro-1H-pyrazol-3-amine,
1-(2-chlorophenyl)-4,5-dihydro-1H-pyrazol-3-amine,
5-ethyl-1-(3-fluorophenyl)-4,5-dihydro-1H-pyrazol-3-amine,
1-(2-chlorophenyl)-4-methyl-4,5-dihydro-1H-pyrazol-3-amine,
1-pyridin-2-yl-4,5-dihydro-1H-pyrazol-3-amine,
1-(4-chlorophenyl)-4,5-dihydro-1H-pyrazol-3-amine, and
5-methyl-1-[3-(trifluoromethyl)phenyl]-4,5-dihydro-1H-pyrazol-3-amine.

As appreciated by those of skill in the art, halo or halogen as used herein are intended to include fluoro, chloro, bromo and iodo. Similarly, C₁₄, as in C₁₋₆allyl, is defined to identify the group as having 1, 2, 3, 4, 5 or 6 carbons in a linear or branched arrangement, such that C₁₋₆alkyl specifically includes methyl, ethyl, n-propyl, iso-propyl, n-butyl, iso-butyl, tert-butyl, pentyl and hexyl. Derived expressions, such as C₁₋₆alkoxy, hydroxyC₁₋₆alkyl and the like are to be construed analogously.

As used herein, aromatic heterocyclic moieties (i.e. “heteroaryl”) include furanyl, imidazolyl, indolyl, indazolyl, isobenzofuranyl, isoindolyl, isoquinolyl, isothiazolyl, isoxazolyl, oxadiazolyl, pyrazinyl, pyrazolyl, pyridazinyl, pyridyl, pyrimidyl, pyrrolyl, quinazolinyl, quinolyl, quinoxalinyl, tetrazolyl, thiadiazolyl, thiazolyl, thienyl, triazolyl, and N-oxides thereof.

Specific embodiments of the present invention include a compound which is selected from the group consisting of the subject compounds of the examples herein and salts thereof and, where appropriate, individual enantiomers and diastereomers thereof.

The compounds of the present invention may contain one or more chiral centers depending on the nature of any substituents present, and can thus occur as racemates and racemic mixtures, single enantiomers, diastereomeric mixtures and individual diastereomers. It is intended that all of the possible optical isomers and diastereomers in mixtures and as pure or partially purified compounds are included within the ambit of this invention. The present invention is meant to comprehend all such isomeric forms of these compounds. Formula I shows the structure of the class of compounds without preferred stereochemistry.

The salts of the present invention are preferably pharmaceutically acceptable. The term “pharmaceutically acceptable salts” refers to salts prepared from pharmaceutically acceptable non-toxic bases or acids including inorganic or organic bases and inorganic or organic acids. Salts in the solid form may exist in more than one crystal structure, and may also be in the form of hydrates. Salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, fumaric, and tartaric acids. It will be understood that, as used herein, references to the compounds of the present invention are meant to also include the pharmaceutically acceptable salts. In addition, salts of the compounds of the formula (I) may be valuable intermediates in preparation of other compounds of the formula (I).

The subject compounds are useful in a method of inhibiting D-amino acid oxidase activity in a patient such as a mammal in need of such inhibition comprising the administration of an effective amount of the compound. The present invention is directed to the use of the compounds disclosed herein as inhibitors of D-amino acid oxidase. In addition to primates, especially humans, a variety of other mammals can be treated according to the method of the present invention.

The present invention is further directed to a method for the manufacture of a medicament for inhibiting D-amino acid oxidase activity in humans and animals which comprises combining a compound of the present invention with a pharmaceutical carrier or diluent.

The subject treated in the present methods is generally a mammal, preferably a human being, male or female, in whom inhibition of D-amino acid oxidase activity is desired. The term “therapeutically effective amount” means the amount of the subject compound that will elicit the biological or medical response of a tissue, system, animal or human that is being sought by the researcher, veterinarian, medical doctor or other clinician. It is recognized that one skilled in the art may affect the neurological and psychiatric disorders by treating a patient presently afflicted with the disorders or by prophylactically treating a patient afflicted with such disorders with an effective amount of the compound of the present invention. As used herein, the terms “treatment” and “treating” refer to all processes wherein there may be a slowing, interrupting, arresting, controlling, or stopping of the progression of the neurological and psychiatric disorders described herein, but does not necessarily indicate a total elimination of all disorder symptoms, as well as the prophylactic therapy to retard the progression or reduce the risk of the noted conditions, particularly in a patient who is predisposed to such disease or disorder.

The terms “administration of” and or “administering a” compound should be understood to mean providing a compound of the invention or a prodrug of a compound of the invention to the individual in need of treatment.

The utility of the compounds in accordance with the present invention as inhibiting D-amino acid oxidase activity may be demonstrated by methodology known in the art.

The compounds of the present invention have utility in treating a variety of neurological and psychiatric disorders associated with glutamatergic neurotransmission dysfunction, including one or more of the following conditions or diseases: schizophrenia or psychosis including schizophrenia (paranoid, disorganized, catatonic or undifferentiated), schizophreniform disorder, schizoaffective disorder, delusional disorder, brief psychotic disorder, shared psychotic disorder, psychotic disorder due to a general medical condition and substance-induced or drug-induced (phencyclidine, ketamine and other dissociative anaesthetics, amphetamine and other psychostimulants and cocaine) psychosispsychotic disorder, psychosis associated with affective disorders, brief reactive psychosis, schizoaffective psychosis, “schizophrenia-spectrum” disorders such as schizoid or schizotypal personality disorders, or illness associated with psychosis (such as major depression, manic depressive (bipolar) disorder, Alzheimer's disease and post-traumatic stress syndrome), including both the positive and the negative symptoms of schizophrenia and other psychoses; cognitive disorders including dementia (associated with Alzheimer's disease, ischemia, multi-infarct dementia, trauma, vascular problems or stroke, HIV disease, Parkinson's disease, Huntington's disease, Pick's disease, Creutzfeldt-Jacob disease, perinatal hypoxia, other general medical conditions or substance abuse); delirium, amnestic disorders or age related cognitive decline; anxiety disorders including acute stress disorder, agoraphobia, generalized anxiety disorder, obsessive-compulsive disorder, panic attack, panic disorder, post-traumatic stress disorder, separation anxiety disorder, social phobia, specific phobia, substance-induced anxiety disorder and anxiety due to a general medical condition; substance-related disorders and addictive behaviors (including substance-induced delirium, persisting dementia, persisting amnestic disorder, psychotic disorder or anxiety disorder; tolerance, dependence or withdrawal from substances including alcohol, amphetamines, cannabis, cocaine, hallucinogens, inhalants, nicotine, opioids, phencyclidine, sedatives, hypnotics or anxiolytics); obesity, bulimia nervosa and compulsive eating disorders; bipolar disorders, mood disorders including depressive disorders; depression including unipolar depression, seasonal depression and post-partum depression, premenstrual syndrome (PMS) and premenstrual dysphoric disorder (PDD), mood disorders due to a general medical condition, and substance-induced mood disorders; learning disorders, pervasive developmental disorder including autistic disorder, attention disorders including attention-deficit hyperactivity disorder (ADHD) and conduct disorder, NMDA receptor-related disorders such as autism, depression, benign forgetfulness, childhood learning disorders and closed head injury; movement disorders, including akinesias and akinetic-rigid syndromes (including Parkinson's disease, drug-induced parkinsonism, postencephalitic parkinsonism, progressive supranuclear palsy, multiple system atrophy, corticobasal degeneration, parkinsonism-ALS dementia complex and basal ganglia calcification), medication-induced parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor), Gilles de la Tourette's syndrome, epilepsy, muscular spasms and disorders associated with muscular spasticity or weakness including tremors; dyskinesias [including tremor (such as rest tremor, postural tremor and intention tremor), chorea (such as Sydenham's chorea, Huntington's disease, benign hereditary chorea, neuroacanthocytosis, symptomatic chorea, drug-induced chorea and hemiballism), myoclonus (including generalised myoclonus and focal myoclonus), tics (including simple tics, complex tics and symptomatic tics), and dystonia (including generalised dystonia such as iodiopathic dystonia, drug-induced dystonia, symptomatic dystonia and paroxymal dystonia, and focal dystonia such as blepharospasm, oromandibular dystonia, spasmodic dysphonia, spasmodic torticollis, axial dystonia, dystonic writer's cramp and hemiplegic dystonia)]; urinary incontinence; neuronal damage including ocular damage, retinopathy or macular degeneration of the eye, tinnitus, hearing impairment and loss, and brain edema; emesis; and sleep disorders including insomnia and narcolepsy.

Of the disorders above, the treatment of schizophrenia, bipolar disorder, depression including unipolar depression, seasonal depression and post-partum depression, premenstrual syndrome (PMS) and premenstrual dysphoric disorder (PDD), learning disorders, pervasive developmental disorder including autistic disorder, attention disorders including Attention-Deficit/Hyperactivity Disorder, autism, tic disorders including Tourette's disorder, anxiety disorders including phobia and post traumatic stress disorder, cognitive disorders associated with dementia, AIDS dementia, Alzheimer's, Parkinson's, Huntington's disease, spasticity, myoclonus, muscle spasm, tinnitus and hearing impairment and loss are of particular importance.

In a specific embodiment, the present invention provides a method for treating cognitive disorders, comprising: administering to a patient in need thereof an effective amount of a compound of the present invention. Particular cognitive disorders are dementia, delirium, amnestic disorders and age-related cognitive decline. At present, the text revision of the fourth edition of the Diagnostic and Statistical Manual of Mental Disorders (DSM-IV-TR) (2000, American Psychiatric Association, Washington D.C.) provides a diagnostic tool that includes cognitive disorders including dementia, delirium, amnestic disorders and age-related cognitive decline. As used herein, the term “cognitive disorders” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “cognitive disorders” is intended to include like disorders that are described in other diagnostic sources.

In another specific embodiment, the present invention provides a method for treating anxiety disorders, comprising: administering to a patient in need thereof an effective amount of a compound of the present invention. Particular anxiety disorders are generalized anxiety disorder, obsessive-compulsive disorder and panic attack. At present, the text revision of the fourth edition of the Diagnostic and Statistical Manual of Mental Disorders (DSM-IV-TR) (2000, American Psychiatric Association, Washington D.C.) provides a diagnostic tool that includes anxiety disorders are generalized anxiety disorder, obsessive-compulsive disorder and panic attack. As used herein, the term “anxiety disorders” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “anxiety disorders” is intended to include like disorders that are described in other diagnostic sources.

In another specific embodiment, the present invention provides a method for treating schizophrenia or psychosis comprising. Administering to a patient in need thereof an effective amount of a compound of the present invention. Particular schizophrenia or psychosis pathologies are paranoid, disorganized, catatonic or undifferentiated schizophrenia and substance-induced psychotic disorder. At present, the text revision of the fourth edition of the Diagnostic and Statistical Manual of Mental Disorders (DSM-IV-TR) (2000, American Psychiatric Association, Washington D.C.) provides a diagnostic tool that includes paranoid, disorganized, catatonic or undifferentiated schizophrenia and substance-induced psychotic disorder. As used herein, the term “schizophrenia or psychosis” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “schizophrenia or psychosis” is intended to include like disorders that are described in other diagnostic sources.

In another specific embodiment, the present invention provides a method for treating substance-related disorders and addictive behaviors, comprising: administering to a patient in need thereof an effective amount of a compound of the present invention. Particular substance-related disorders and addictive behaviors are persisting dementia, persisting amnestic disorder, psychotic disorder or anxiety disorder induced by substance abuse; and tolerance of, dependence on or withdrawal from substances of abuse. At present, the text revision of the fourth edition of the Diagnostic and Statistical Manual of Mental Disorders (DSM-IV-TR) (2000, American Psychiatric Association, Washington D.C.) provides a diagnostic tool that includes persisting dementia, persisting amnestic disorder, psychotic disorder or anxiety disorder induced by substance abuse; and tolerance of, dependence on or withdrawal from substances of abuse. As used herein, the term “substance-related disorders and addictive behaviors” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “substance-related disorders and addictive behaviors” is intended to include like disorders that are described in other diagnostic sources.

The compounds of the present invention will be of use in the prevention or treatment of diseases and conditions in which pain and/or inflammation predominates, including chronic and acute pain conditions. Such conditions include rheumatoid arthritis; osteoarthritis; post-surgical pain; musculoskeletal pain, particularly after trauma; spinal pain; myofascial pain syndromes; headache, including migraine, acute or chronic tension headache, cluster headache, temporomandibular pain, and maxillary sinus pain; ear pain; episiotomy pain; burns, and especially primary hyperalgesia associated therewith; deep and visceral pain, such as heart pain, muscle pain, eye pain, orofacial pain, for example, odontalgia, abdominal pain, gynaecological pain, for example, dysmenorrhoea, pain associated with cystitis and labour pain; pain associated with nerve and root damage, such as pain associated with peripheral nerve disorders, for example, nerve entrapment and brachial plexus avulsions, amputation, peripheral neuropathies, tic douloureux, atypical facial pain, nerve root damage, and arachnoiditis; itching conditions including pruritis, itch due to hemodialysis, and contact dermatitis; pain (as well as broncho-constriction and inflammation) due to exposure (e.g. via ingestion, inhalation, or eye contact) of mucous membranes to capsaicin and related irritants such as tear gas, hot peppers or pepper spray; neuropathic pain conditions such as diabetic neuropathy, chemotherapy-induced neuropathy and post-herpetic neuralgia; “non-painful” neuropathies; complex regional pain syndromes; pain associated with carcinoma, often referred to as cancer pain; central nervous system pain, such as pain due to spinal cord or brain stem damage, low back pain, sciatica and ankylosing spondylitis; gout; scar pain; irritable bowel syndrome; inflammatory bowel disease; urinary incontinence including bladder detrusor hyper-reflexia and bladder hypersensitivity; respiratory diseases including chronic obstructive pulmonary disease (COPD), chronic bronchitis, cystic fibrosis and asthma; autoimmune diseases; and immunodeficiency disorders.

In another specific embodiment, the present invention provides a method for treating pain, comprising: administering to a patient in need thereof an effective amount of a compound of the formula (I) or a composition comprising a compound of formula (I). Particular pain embodiments are bone and joint pain (osteoarthritis), repetitive motion pain, dental pain, cancer pain, myofascial pain (muscular injury, fibromyalgia), perioperative pain (general surgery, gynecological), chronic pain and neuropathic pain.

According to a further or alternative aspect, the present invention provides a compound of formula (I) for use in the manufacture of a medicament for the treatment or prevention of a disease or condition in which pain and/or inflammation predominates.

In another specific embodiment, the present invention provides a method for treating obesity or eating disorders associated with excessive food intake and complications associated therewith, comprising: administering to a patient in need thereof an effective amount of a compound of the present invention. At present, obesity is included in the tenth edition of the International Classification of Diseases and Related Health Problems (ICD-10) (1992 World Health Organization) as a general medical condition. The text revision of the fourth edition of the Diagnostic and Statistical Manual of Mental Disorders (DSM-IV-TR) (2000, American Psychiatric Association, Washington D.C.) provides a diagnostic tool that includes obesity in the presence of psychological factors affecting medical condition. As used herein, the term “obesity or eating disorders associated with excessive food intake” includes treatment of those medical conditions and disorders described in ICD-10 and DSM-IV-TR. The skilled artisan will recognize that there are alternative nomenclatures, nosologies and classification systems for general medical conditions, and that these systems evolve with medical and scientific progress. Thus the term “obesity or eating disorders associated with excessive food intake” is intended to include like conditions and disorders that are described in other diagnostic sources.

The subject compounds are further useful in a method for the prevention, treatment, control, amelioration, or reducation of risk of the diseases, disorders and conditions noted herein.

The subject compounds are further useful in a method for the prevention, treatment, control, amelioration, or reduction of risk of the aforementioned diseases, disorders and conditions in combination with other agents, including an inhibitor of glycine transporter GlyT1 activity

The compounds of the present invention may be used in combination with one or more other drugs in the treatment, prevention, control, amelioration, or reduction of risk of diseases or conditions for which compounds of the present invention or the other drugs may have utility, where the combination of the drugs together are safer or more effective than either drug alone. Such other drug(s) may be administered, by a route and in an amount commonly used therefor, contemporaneously or sequentially with a compound of the present invention. When a compound of the present invention is used contemporaneously with one or more other drugs, a pharmaceutical composition in unit dosage form containing such other drugs and the compound of the present invention is preferred. However, the combination therapy may also include therapies in which the compound of the present invention and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of the present invention.

The above combinations include combinations of a compound of the present invention not only with one other active compound, but also with two or more other active compounds. Likewise, compounds of the present invention may be used in combination with other drugs that are used in the prevention, treatment, control, amelioration, or reduction of risk of the diseases or conditions for which compounds of the present invention are useful. Such other drugs may be administered, by a route and in an amount commonly used therefor, contemporaneously or sequentially with a compound of the present invention. When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

The weight ratio of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

Accordingly, the subject compounds may be used alone or in combination with other agents which are known to be beneficial in the subject indications or other drugs that affect receptors or enzymes that either increase the efficacy, safety, convenience, or reduce unwanted side effects or toxicity of the compounds of the present invention. The subject compound and the other agent may be co-administered, either in concomitant therapy or in a fixed combination.

In one embodiment, the subject compound may be employed in combination with anti-Alzheimer's agents, beta-secretase inhibitors, gamma-secretase inhibitors, HMG-CoA reductase inhibitors, NSAID's including ibuprofen, vitamin E, and anti-amyloid antibodies.

In another embodiment, the subject compound may be employed in combination with sedatives, hypnotics, anxiolytics, antipsychotics, cyclopyrrolones, imidazopyridines, pyrazolopyrimidines, minor tranquilizers, melatonin agonists and antagonists, melatonergic agents, benzodiazepines, barbiturates, 5HT-2 antagonists, and the like, such as: adinazolam, allobarbital, alonimid, alprazolam, amisulpride, amitriptyline, amobarbital, amoxapine, aripiprazole, bentazepam, benzoctamine, brotizolam, bupropion, busprione, butabarbital, butalbital, capuride, carbocloral, chloral betaine, chloral hydrate, clomipramine, clonazepam, cloperidone, clorazepate, chlordiazepoxide, clorethate, chlorpromazine, clozapine, cyprazepam, desipramine, dexclamol, diazepam, dichloralphenazone, divalproex, diphenhydramine, doxepin, estazolam, ethchlorvynol, etomidate, fenobam, flunitrazepam, flupentixol, fluphenazine, flurazepam, fluvoxamine, fluoxetine, fosazepam, glutethimide, halazepam, haloperidol, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, olanzapine, oxazepam, paraldehyde, paroxetine, pentobarbital, perlapine, perphenazine, phenelzine, phenobarbital, prazepam, promethazine, propofol, protriptyline, quazepam, quetiapine, reclazepam, risperidone, roletamide, secobarbital, sertraline, suproclone, temazepam, thioridazine, thiothixene, tracazolate, tranylcypromaine, trazodone, triazolam, trepipam, tricetamide, triclofos, trifluoperazine, trimetozine, trimipramine, uldazepam, venlafaxine, zaleplon, ziprasidone, zolazepam, zolpidem, and salts thereof, and combinations thereof, and the like, or the subject compound may be administered in conjunction with the use of physical methods such as with light therapy or electrical stimulation.

In another embodiment, the subject compound may be employed in combination with levodopa (with or without a selective extracerebral decarboxylase inhibitor such as carbidopa or benserazide), anticholinergics such as biperiden (optionally as its hydrochloride or lactate salt) and trihexyphenidyl (benzhexol)hydrochloride, COMT inhibitors such as entacapone, MOA-B inhibitors, antioxidants, A2a adenosine receptor antagonists, cholinergic agonists, NMDA receptor antagonists, serotonin receptor antagonists and dopamine receptor agonists such as alentemol, bromocriptine, fenoldopam, lisuride, naxagolide, pergolide and pramipexole. It will be appreciated that the dopamine agonist may be in the form of a pharmaceutically acceptable salt, for example, alentemol hydrobromide, bromocriptine mesylate, fenoldopam mesylate, naxagolide hydrochloride and pergolide mesylate. Lisuride and pramipexol are commonly used in a non-salt form.

In another embodiment, the subject compound may be employed in combination with a compound from the phenothiazine, thioxanthene, heterocyclic dibenzazepine, butyrophenone, diphenylbutylpiperidine and indolone classes of neuroleptic agent. Suitable examples of phenothiazines include chlorpromazine, mesoridazine, thioridazine, acetophenazine, fluphenazine, perphenazine and trifluoperazine. Suitable examples of thioxanthenes include chlorprothixene and thiothixene. An example of a dibenzazepine is clozapine. An example of a butyrophenone is haloperidol. An example of a diphenylbutylpiperidine is pimozide. An example of an indolone is molindolone. Other neuroleptic agents include loxapine, sulpiride and risperidone. It will be appreciated that the neuroleptic agents when used in combination with the subject compound may be in the form of a pharmaceutically acceptable salt, for example, chlorpromazine hydrochloride, mesoridazine besylate, thioridazine hydrochloride, acetophenazine maleate, fluphenazine hydrochloride, flurphenazine enathate, fluphenazine decanoate, trifluoperazine hydrochloride, thiothixene hydrochloride, haloperidol decanoate, loxapine succinate and molindone hydrochloride. Perphenazine, chlorprothixene, clozapine, haloperidol, pimozide and risperidone are commonly used in a non-salt form. Thus, the subject compound may be employed in combination with acetophenazine, alentemol, aripiprazole, amisulpride, benzhexol, bromocriptine, biperiden, chlorpromazine, chlorprothixene, clozapine, diazepam, fenoldopam, fluphenazine, haloperidol, levodopa, levodopa with benserazide, levodopa with carbidopa, lisuride, loxapine, mesoridazine, molindolone, naxagolide, olanzapine, pergolide, perphenazine, pimozide, pramipexole, quetiapine, risperidone, sulpiride, tetrabenazine, trihexyphenidyl, thioridazine, thiothixene, trifluoperazine or ziprasidone.

In another embodiment, the subject compound may be employed in combination with an anti-depressant or anti-anxiety agent, including norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, neurokinin-1 receptor antagonists, atypical anti-depressants, benzodiazepines, 5-HT_{1A} agonists or antagonists, especially 5-HT_{1A} partial agonists, and corticotropin releasing factor (CRF) antagonists. Specific agents include: amitriptyline, clomipramine, doxepin, imipramine and trimipramine; amoxapine, desipramine, maprotiline, nortriptyline and protriptyline; fluoxetine, fluvoxamine, paroxetine and sertraline; isocarboxazid, phenelzine, tranylcypromine and selegiline; moclobemide: venlafaxine; duloxetine; aprepitant; bupropion, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone, and pharmaceutically acceptable salts thereof.

In another embodiment, the subject compound may be employed in combination with a compound useful in the treatment of pain, for example an NSAID such as ibuprofen, an antinociceptive agent such as an NR2B antagonist, a COX2 inhibitor such as ARCOXIA or a sodium channel blocker.

The compounds of the present may also be employed in combination with D-amino acids or suitable derivatives thereof such as D-phenylalanine, parafluoro-D-phenyl alanine, D-(N-trifluoroacetyl-4-fluorophenylalanine), D-leucine, D-alanine, D-cycloserine and D-serine or D/L mixtures thereof.

Preferred combinations of the present invention include compounds of the formula (I) in combination with D-serine, clozapine, haloperidole, olanzapine, or risperidone.

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

The term “composition” as used herein is intended to encompass a product comprising specified ingredients in predetermined amounts or proportions, as well as any product which results, directly or indirectly, from combination of the specified ingredients in the specified amounts. This term in relation to pharmaceutical compositions is intended to encompass a product comprising one or more active ingredients, and an optional carrier comprising inert ingredients, as well as any product which results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. Accordingly, the pharmaceutical compositions of the present invention encompass any composition made by admixing a compound of the present invention and a pharmaceutically acceptable carrier.

Pharmaceutical compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients that are suitable for the manufacture of tablets. The tablets may be uncoated or they may be coated by known techniques to delay disintegration and absorption in the gastrointestinal tract and thereby provide a sustained action over a longer period or may be tablets that disperse when added to water. Compositions for oral use may also be presented as hard gelatin capsules wherein the active ingredients are mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Aqueous suspensions, oily suspensions, dispersible powders or granules, oil-in-water emulsions, and sterile injectable aqueous or oleagenous suspension may be prepared by standard methods known in the art.

In the treatment of conditions which require inhibition of D-amino acid oxidase activity an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900, and 1000 milligrams of the active ingredient for the symptomatic adjustment of the dosage to the patient to be treated. The compounds may be administered on a regimen of 1 to 4 times per day, preferably once or twice per day. This dosage regimen may be adjusted to provide the optimal therapeutic response. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Several methods for preparing the compounds of this invention are illustrated in the following Schemes and Examples. Starting materials and the requisite intermediates are in some cases commercially available, or can be prepared according to literature procedures or as illustrated herein.

The compounds of this invention may be prepared by the methods disclosed in EP 0 070376, EP 0 127 371 and in Tetrahedron Letters, 1998, 39, 5845-8, or adaptations thereof, and as described hereinafter.

For example, the reaction of a compound of the formula (IV):

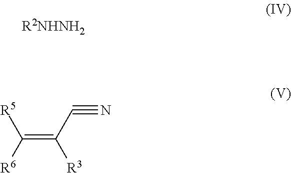

with a compound of the formula (V):
and R¹ to R⁶ are as hereinbefore defined, provides a compound of formula (I) wherein R¹, R^{1a} and R⁴ are hydrogen.

The reaction of a compound of the formula (IV) with a compound of the formula (V) is conveniently carried out in the presence of a base, for example an alkyl metal base such as butyl lithium or a metal alkoxide base such as sodium ethoxide, in a nonreactive solvent, for example a C₁₋₆alkanol such as ethanol, at a non-extreme temperature, for example 20° C. to 150° C., suitably 50° C. to 100° C., and conveniently at the reflux temperature of the solvent. Alternatively the reaction can be facilitated by microwave energy when the reaction will be at an elevated temperature for example 50° C. to 150° C. and conveniently 90° C. to 150° C.

The independent syntheses of diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

In some cases the final product may be further modified, for example, by manipulation of substituents. These manipulations may include, but are not limited to, reduction, oxidation, alkylation, acylation, and deesterification/hydrolysis reactions which are commonly known to those skilled in the art. Certain compounds of the formula (I) may therefore be useful as intermediates in preparation of other compounds of the formula (I). For example, the 3-amino group in compounds of formula (I) in which R′ and R^{1a} are both H may by alkylated, acylated or sulfonated by conventional techniques. In some cases the order of carrying out the foregoing reaction schemes may be varied to facilitate the reaction or to avoid unwanted reaction products. The following examples are provided so that the invention might be more fully understood. These examples are illustrative only and should not be construed as limiting the invention in any way.

### EXAMPLE 1

### 1-(2-Fluorophenyl)-5,5-dimethyl-4,5-dihydro-1H-pyrazol-3-amine

To a solution of EtONa (8.0 mL, 1.0 mol/L) in ethanol (5.0 mL) (2-fluorophenyl)hydrazine (2.0 mmol) was added and stirred for 10 minutes followed by addition of 3-methylbut-2-enenitrile (2.0 mmol). The mixture was refluxed overnight (16 hours), and then concentrated in vacuo. Water (10 mL) was added to the residue and this was extracted with CH₂Cl₂ (10×3 mL). The organic phase was dried over MgSO₄. After removal of ⅔ solvent in vacuo, hexane was added, the resultant slurry filtered and the solid obtained washed with hexane. After being dried under vacuum, 1-(2-fluorophenyl)-5,5-dimethyl-4,5-dihydro-1H-pyrazol-3-amine was obtained. (300 MHz, MeOD) δ 7.16˜7.31 (m, 4H), 3.07 (s, 2H), 1.29 (s, 6H).

### EXAMPLES 2-25

Starting with the appropriate hydrazine and nitrile, the following compounds were prepared by a method analogous to example 1.

5-(4-chlorophenyl)-1-(2-fluorophenyl)-4,5-dihydro-1H-pyrazol-3-amine, (300 MHz, DMSO) δ 7.28˜7.70 (br, 8H), 5.19 (br, 2H), 3.64 (br, 1H), 2.78 (br, 1H),
1-(3-fluorophenyl)-5-methyl-4,5-dihydro-1H-pyrazol-3-amine, (300 MHz, MeOD) δ 6.50˜6.75 (m, 4H), 4.18 (br, 1H), 3.53 (br, 1H), 1.48 (s, 3H),
1-(2-fluorophenyl)-5-phenyl-4,5-dihydro-1H-pyrazol-3-amine, (400 MHz, DMSO) δ 7.30˜7.39 (m, 6H), 7.08˜7.12 (m, 2H), 6.96˜6.99 (m, 1H), 5.09 (d, J=9.2 Hz, 1H), 3.68 (d-d, J=16.2 Hz, 9.2 Hz, 1H), 2.49 (d-d, J=16.2 Hz, 2.8 Hz, 1H),
1-(3-chlorophenyl)-5-methyl-4,5-dihydro-1H-pyrazol-3-amine, (400 MHz, MeOD) δ 7.07˜7.34 (m, 4H), 4.12 (br, 1H), 3.48 (br, 1H), 2.70 (br, 1H), 1.42 (d, J=6.0 Hz, 3H),
1-(4-chlorophenyl)-5-methyl-4,5-dihydro-1H-pyrazol-3-amine, (400 MHz, D2O) δ 7.01 (d, J=8.0 Hz, 2 H), 6.86 (d, J=8.0 Hz, 2H), 3.93 (br, 1H), 3.31 (d-d, J=17.6 Hz, 8.0 Hz, 1H), 2.62-2.66 (m, 2H), 1.26 (d, J=6.4 Hz, 3H),
5-ethyl-1-(2-fluorophenyl)-4,5-dihydro-1H-pyrazol-3-amine, (400 MHz, MeOD) δ 7.50 (br, 4H), 4.34 (br, 1H), 3.80˜3.98 (m, 1H), 3.24˜3.28 (m, 1H), 2.20˜2.37 (m, 2H), 1.58 (t, J=7.2 Hz, 3H),
1-(3-chlorophenyl)-4-methyl-4,5-dihydro-1H-pyrazol-3-amine, (300 MHz, MeOD) δ 7.06˜7.11 (m, 1H), 6.88 (s, 1H), 6.61˜2.70 (m, 2H), 3.70 (br, 1H), 3.13˜3.15 (m, 2H), 1.27 (d, J=6.9 Hz, 3H), 1-(3-fluorophenyl)-4,5-dihydro-1H-pyrazol-3-amine,
5-(4-fluorophenyl)-1-pyridin-3-yl-4,5-dihydro-1H-pyrazol-3-amine, (300 MHz, MeOD) δ 8.07 (s, 1H), 7.72˜7.75 (m, 1H), 7.46˜7.58 (m, 2H), 7.31˜7.38 (m, 2H), 7.07˜7.19 (m, 2H), 5.30˜5.36 (m, 1H), 3.65 (d-d, J=16.8 Hz, 6.0 Hz, 1H), 2.79 (d-d, J=16.8 Hz, 4.5 Hz, 1H),
1-(4-chlorophenyl)-5-phenyl-4,5-dihydro-1H-pyrazol-3-amine, (400 MHz, MeOD) δ 7.23˜7.42 (m, 5H), 6.98˜7.00 (m, 2H), 6.69˜6.72 (m, 2H), 4.81˜4.92 (m, 1H), 3.51 (dd, J=14.8 Hz, 7.2 Hz, 1H), 2.64˜2.70 (m, 1H),
1-(3-chlorophenyl)-5-phenyl-4,5-dihydro-1H-pyrazol-3-amine, (400 MHz, MeOD) δ 7.28˜7.36 (m, 5H), 6.93˜6.97 (m, 1H), 6.80 (s, 1H), 6.53˜6.55 (m, 2H), 4.94 (br, 1H), 3.49˜3.56 (m, 1H), 2.68 (d-d, J=16.0 Hz, 6.4 Hz, 1H),
1-(2-fluorophenyl)-4-methyl-4,5-dihydro-1H-pyrazol-3-amine, (400 MHz, MeOD) δ7.13˜7.28 (m, 4H), 4.01˜4.04 (m, 1H), 3.46˜3.53 (m, 2H), 1.32 (d, 3H),
1-(3-chlorophenyl)-4,5-dihydro-1H-pyrazol-3-amine, (400 MHz, MeOD) δ6.64˜7.43 (m, 4H), 3.89 (br, 2H), 2.95 (br, 2H),
4-methyl-1-pyridin-2-yl-4,5-dihydro-1H-pyrazol-3-amine, (400 MHz, MeOD) δ 6.55˜8.12 (m, 4H), 4.20 (t, 1H), 4.03 (br, 2H), 1.32 (d, 3H),
5-ethyl-1-pyridin-2-yl-4,5-dihydro-1H-pyrazol-3-amine, (400 MHz, MeOD) δ 6.62˜7.78 (m, 4H), 4.53˜4.60 (m, 1H), 3.32, 3.67 (2dd, 1H), 2.76, 2.81 (2dd, 1H), 1.65˜1.80 (m, 2H), 0.98 (t, J=6.8 Hz, 3H),
1-(3-fluorophenyl)-5-(trifluoromethyl)-4,5-dihydro-1H-pyrazol-3-amine, (400 MHz, MeOD) δ 6.74˜7.34 (m, 4H), 4.71˜4.81 (m, 1H), 3.68, 3.74 (2dd, 1H), 3.06, 3.12 (2dd, 1H),
1-(2-fluorophenyl)-4,5-dihydro-1H-pyrazol-3-amine, (300 MHz, CDCl3) δ 7.003˜7.80 (m, 4H), 3.88 (br, 1H), 3.19 (br, 1H),
1-(2-chlorophenyl)-4,5-dihydro-1H-pyrazol-3-amine, (300 MHz, CDCl3) δ 6.91 (m, 4H), 4.19 (br, 1H), 3.74 (t, J=6.9 Hz, 2H), 2.87 (t, J=6.9 Hz, 2H),
5-ethyl-1-(3-fluorophenyl)-4,5-dihydro-1H-pyrazol-3-amine, (400 MHz, CDCl3) δ 6.88˜7.40 (m, 4H), 4.59 (br, 2H), 4.04˜4.59 (m, 1H), 3.07˜3.15 (m, 2H), 1.20 (d, 3H),
1-(2-chlorophenyl)-4-methyl-4,5-dihydro-1H-pyrazol-3-amine, (400 MHz, CDCl3) δ 7.45˜8.60 (m, 4H), 3.53˜3.78 (m, 1H), 2.60, 3.29 (dd, 2H), 1.49˜1.79 (m, 2H), 0.93˜1.25 (m, 3H),
1-pyridin-2-yl-4,5-dihydro-1H-pyrazol-3-amine, (400 MHz, CDCl3) δ 6.55˜8.17 (m, 4H), 3.98˜4.28 (dd, 4H), 2.82˜2.93 (m, 2H), and
1-(4-chlorophenyl)-4,5-dihydro-1H-pyrazol-3-amine, (400 MHz, MeOD) δ 7.12 (d, J=7.2 Hz, 2H), 6.92 (d, J=7.2 Hz, 2H), 3.66 (t, J=8.8 Hz, 2H), 2.87 (t, J=8.8 Hz, 2H).

### EXAMPLE 26

### 5-Methyl-1-pyridin-2-yl-4,5-dihydro-1H-pyrazol-3-amine

Sodium metal (15.0 mg, 0.65 mmol) was dissolved in 4.0 ml of absolute ethanol, then 2-hydrazinopyridine (327.3 mg, 3.0 mmol) was added, followed by (2E)-but-2-enenitrile (246 μl, 3 mmol). The reaction mixture was refluxed overnight. The solvent was removed in vacuo and water added to the residue to separate a solid. The solid was collected by filtration, dissolved in dichloromethane, dried over magnesium sulfate, filtered and concentrated to one-third of the original volume. Hexane was added to the residue so that crystals separated out of solution. The mixture was cooled and filtered to give 5-methyl-1-pyridin-2-yl-4,5-dihydro-1H-pyrazol-3-amine (210 mg, yield 39.7%).

### EXAMPLE 27

### 5-Methyl-1-[3-(trifluoromethyl)phenyl]-4,5-dihydro-1H-pyrazol-3-amine

To a solution of EtONa (0.2 mL, 1.0 mol/L) in ethanol (2.0 mL), [3-(trifluoromethyl)phenyl]hydrazine (176.1 mg, 1.0 mmol) was added, followed by (2E)-but-2-enenitrile (82 microL, 1 mmol). The mixture was reacted under microwave condition (120° C., 30 min). The mixture was then concentrated in vacuo. Water (5 mL) was added and the mixture extracted with CH₂Cl₂ (10 mL×2). The residue was purified by preparative HPLC to give 5-methyl-1-[3-(trifluoromethyl)phenyl]-4,5-dihydro-1H-pyrazol-3-amine. (300 MHz, MeOD) δ 7.60˜7.61 (m, 1H), 7.31˜7.41 (m, 3H), 4.18˜4.20 (m, 1H), 3.46˜3.52 (m, 1H), 2.61-2.66 (m, 1H), 1.43 (d, J=6.6 Hz, 3H). The process was repeated at 100° C. for 40 min to give the same product (90 mg, yield 37%).

### ASSAY EXAMPLE

### Cell Based Assay Protocol to Determine Efficacy of D-Amino Acid Oxidase Inhibitors

CHO cells stably expressing human D-amino acid oxidase were grown in F12/Ham glutamax medium containing 10% FBS, 1× pen/strep and 1 mg/ml G418. On the day of assay, cells were washed with PBS, harvested and spun at 1000 rpm for 5 mins before resuspending in assay buffer (HBSS containing 1 M CaCl₂, 1 M MgCl₂ and 1 M Hepes, pH 7.4) at 8.6×10⁵/ml. 35 ul cell suspension was added to 5 ul test compound in a 384 well plate. The assay was initiated by the addition of 10 ul assay buffer containing 2.5% amplex red (Molecular Probes), 6% horse radish peroxidase and 25% 1 M D-serine. Plates were incubated for 2 hours at 37° C. and fluorescence (excitation 544 nm, emission 590 nm) read using a Cytofluor plate reader. Compounds of the present invention had activity at below the one micromolar level.

1-8. (canceled)

9. A compound of the formula (I): 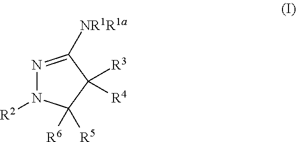
wherein:
R¹ and R^{1a} are independently selected from hydrogen, C₁₋₆alkyl, haloC₁₋₆alkyl, hydroxyC₁₋₆alkyl, C₁₋₆alkylcarbonyl, and a group SO₂R⁷ wherein R⁷ is amino or C₁₋₆alkyl optionally substituted by phenyl;
R² is selected from phenyl, 5- or 6-membered heteroaryl which is optionally benzofused, 5- or 6-membered carbocyclic, and 5- or 6-membered heterocyclic containing at least one hetero atom selected from oxygen, nitrogen and sulphur, each of which may be substituted by C₁₋₆alkyl, C₁₋₆alkoxy, CF₃ or OCF₃;
or R² is C₁₋₆alkyl optionally substituted by hydroxyl, halo or amino optionally substituted by one or two C₁₋₆alkyl groups; or a group (CH₂)ₘphenyl, wherein m is 1 or 2, which may be substituted by halo, hydroxyl, amino optionally substituted by one or two C₁₋₆alkyl groups;
and R³; R⁴, R⁵ and R⁶ are independently selected from hydrogen, CF₃, C₁₋₆alkyl and phenyl each optionally substituted by halo or hydroxyl;
or a pharmaceutically acceptable salt thereof.

10. The compound of claim 9 wherein R¹ and R^{1a} are both hydrogen.

11. The compound of claim 9 wherein R² is phenyl optionally substituted by fluoro, chloro, methoxy, or trifluoromethyl; unsubstituted pyridyl; or pyrazolyl substituted by amino.

12. The compound of claim 9 wherein at least one of R³ and R⁴ is hydrogen.

13. The compound of claim 9 wherein R³, R⁴, R⁵ and R⁶ are selected from hydrogen, methyl, ethyl, trifluoromethyl and phenyl optionally substituted by a fluoro or chloro atom at the para position.

14. A compound which is selected from the group consisting of:
5-(4-chlorophenyl)-1-(2-fluorophenyl)-4,5-dihydro-1H-pyrazol-3-amine,
5-(4-chlorophenyl)-1-(2-fluorophenyl)-4,5-dihydro-1H-pyrazol-3-amine,
1-(3-fluorophenyl)-5-methyl-4,5-dihydro-1H-pyrazol-3-amine,
1-(2-fluorophenyl)-5-phenyl-4,5-dihydro-1H-pyrazol-3-amine,
1-(3-chlorophenyl)-5-methyl-4,5-dihydro-1H-pyrazol-3-amine,
1-(4-chlorophenyl)-5-methyl-4,5-dihydro-1H-pyrazol-3-amine,
5-ethyl-1-(2-fluorophenyl)-4,5-dihydro-1H-pyrazol-3-amine,
1-(3-chlorophenyl)-4-methyl-4,5-dihydro-1H-pyrazol-3-amine,
1-(3-fluorophenyl)-4,5-dihydro-1H-pyrazol-3-amine,
5-(4-fluorophenyl)-1-pyridin-3-yl-4,5-dihydro-1H-pyrazol-3-amine,
1-(4-chlorophenyl)-5-phenyl-4,5-dihydro-1H-pyrazol-3-amine,
1-(4-chlorophenyl)-5-phenyl-4,5-dihydro-1H-pyrazol-3-amine,
1-(3-chlorophenyl)-5-phenyl-4,5-dihydro-1H-pyrazol-3-amine,
1-(2-fluorophenyl)-4-methyl-4,5-dihydro-1H-pyrazol-3-amine,
1-(3-chlorophenyl)-4,5-dihydro-1H-pyrazol-3-amine,
4-methyl-1-pyridin-2-yl-4,5-dihydro-1H-pyrazol-3-amine,
5-ethyl-1-pyridin-2-yl-4,5-dihydro-1H-pyrazol-3-amine,
1-(3-fluorophenyl)-5-(trifluoromethyl)-4,5-dihydro-1H-pyrazol-3-amine,
1-(2-fluorophenyl)-4,5-dihydro-1H-pyrazol-3-amine,
1-(2-chlorophenyl)-4,5-dihydro-1H-pyrazol-3-amine,
5-ethyl-1-(3-fluorophenyl)-4,5-dihydro-1H-pyrazol-3-amine,
1-(2-chlorophenyl)-4-methyl-4,5-dihydro-1H-pyrazol-3-amine,
1-pyridin-2-yl-4,5-dihydro-1H-pyrazol-3-amine,
1-(4-chlorophenyl)-4,5-dihydro-1H-pyrazol-3-amine, and
5-methyl-1-[3-(trifluoromethyl)phenyl]-4,5-dihydro-1H-pyrazol-3-amine;
or a pharmaceutically acceptable salt thereof.

15. A pharmaceutical composition comprising the compound of claim 9, or a pharmaceutically acceptable salt thereof, and a pharmaceutically acceptable carrier.

16. A method of inhibiting D-amino acid oxidase activity in a mammal in need of such inhibition comprising the administration of an effective amount of a compound of claim 9, or a pharmaceutically acceptable salt thereof.
